(19) United States
(12) Patent Application Publication
(10) Pub. No.: US 2020/0230379 A1
(43) Pub. Date: Jul. 23, 2020

(54) MEDICAL INFLATION SYSTEMS AND METHODS

(71) Applicant: Merit Medical Systems, Inc., South Jordan, UT (US)

(72) Inventors: Adam Simmons, South Jordan, UT (US); David Craig Chadwick, West Jordan, UT (US)

(21) Appl. No.: 16/745,116

(22) Filed: Jan. 16, 2020

Related U.S. Application Data
(60) Provisional application No. 62/794,036, filed on Jan. 18, 2019.

(51) Int. Cl. A61M 25/10

(52) U.S. Cl. CPC A61M 25/10182

(57) ABSTRACT

Devices used to pressurize, depressurize, or otherwise displace fluid are disclosed. The devices may be configured to displace fluid in order to inflate or deflate a medical device, such as a balloon. The devices further include a crank member for providing a mechanical advantage when pressurizing or otherwise displacing fluid. The devices further include adding reinforcement fibers and slip agents to polymeric materials.

### RELATED CASES

This application claims priority to U.S. Provisional Application No. 62/794,036, filed on Jan. 18, 2019, and titled “Medical Inflation Systems and Methods,” which is hereby incorporated by reference in its entirety.

### TECHNICAL FIELD

The present disclosure relates generally to devices used to pressurize, depressurize, or otherwise displace fluid, for example in medical devices. More specifically, the present disclosure relates to high-pressure devices used to pressurize, depressurize, or otherwise displace fluid along a line in order to inflate or deflate a medical device, such as a balloon. The disclosure further relates to additives and polymeric materials for providing strength and lubricity to such devices.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a front perspective view of an inflation device assembly.

FIG. 1B is a side view of the inflation device assembly of FIG. 1A.

FIG. 2 is a detail cross-sectional view of the coupling member (shown in engagement with the plunger threads and the plunger surface) of the inflation device of FIG. 1A, taken along plane 2-2 of FIG. 1A.

FIG. 3 is an exploded detail view of the grip and related elements of the inflation device assembly of FIG. 1A.

FIG. 4 is an exploded detail view of a hinge and related components of the inflation device assembly of FIG. 1A.

FIG. 5 is a back perspective view of the inflation device assembly of FIG. 1A.

FIG. 6 is a front side view of the inflation device assembly of FIG. 1A.

FIG. 7 is a back side view of the inflation device assembly of FIG. 1A.

FIG. 8 is a top view of the inflation device assembly of FIG. 1A.

FIG. 9 is a bottom view of the inflation device assembly of FIG. 1A.

FIG. 10 is a left side view of the inflation device assembly of FIG. 1A.

FIG. 11 is a right side view of the inflation device assembly of FIG. 1A.

### DETAILED DESCRIPTION

An inflation device may comprise a syringe which utilizes threads to advance or retract a plunger by rotating the plunger handle relative to the body of the syringe such that the threads cause longitudinal displacement of the plunger relative to the body. In some instances, an inflation syringe may comprise retractable threads, configured to enable a practitioner to disengage the threads and displace the plunger by simply pushing or pulling the plunger.

The inflation syringe may comprise a coupling member configured to constrain movement of the plunger within the syringe body. The coupling member may comprise threads configured to engage with retractable threads. Certain inflation devices include a mechanism in the handle of the device which allows the practitioner to disengage the threads through manipulating the mechanism. For example, in some instances the handle of such a device may include a “trigger” portion configured to retract threads positioned on the plunger. Actuation of the trigger may thus transition the threads between an engaged configuration where the threads are engaged with the coupling member and a released, or disengaged configuration, where the plunger is configured to be displaced with respect to the syringe body by pushing or pulling on the plunger.

In some embodiments, inflation devices within the scope of this disclosure may be used to generate relatively high pressures, for example pressures of more than 30 atmospheres, more than 40 atmospheres, and more than 50 atmospheres, or higher. At higher pressures, displacement of the plunger (either by direct axial displacement or by rotation of the plunger when the threads are engaged) may be difficult due to the forces generated on the plunger by pressure in the syringe.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Further, various features are sometimes grouped together in a single embodiment, figure, or description thereof for the purpose of streamlining the disclosure. Many of these features may be used alone and/or in combination with one another.

The phrase “coupled to” refers to any form of interaction between two or more components, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As specifically applied to the syringe portion of an inflation device, the proximal end of the syringe refers to the end nearest the handle and the distal end refers to the opposite end, the end nearest the inlet/outlet port of the syringe. Thus, if at one or more points in a procedure a practitioner changes the orientation of a syringe, as used herein, the term “proximal end” always refers to the handle end of the syringe (even if the distal end is temporarily closer to the practitioner).

“Fluid” is used in its broadest sense, to refer to any fluid, including both liquids and gases as well as solutions, compounds, suspensions, etc., which generally behave as fluids.

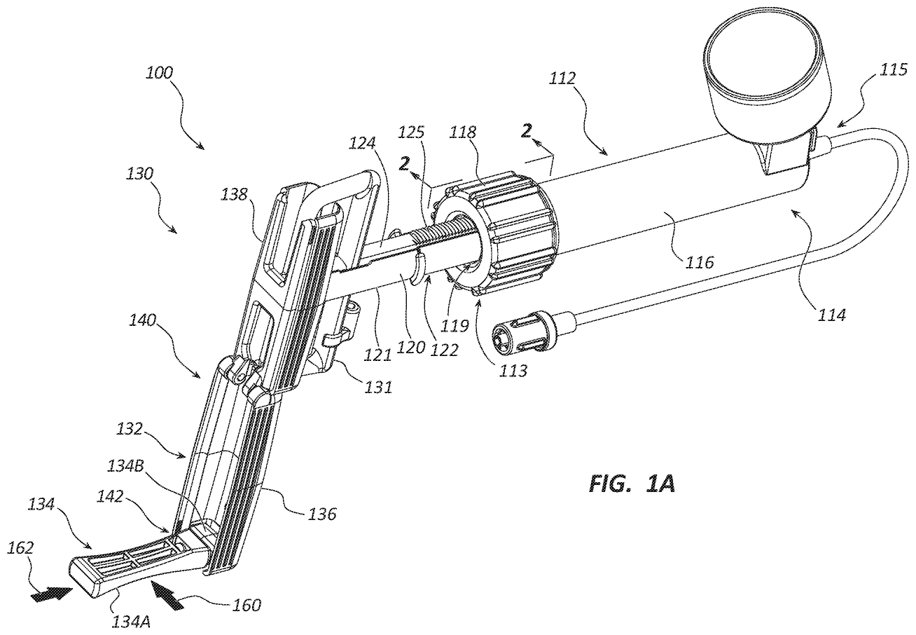

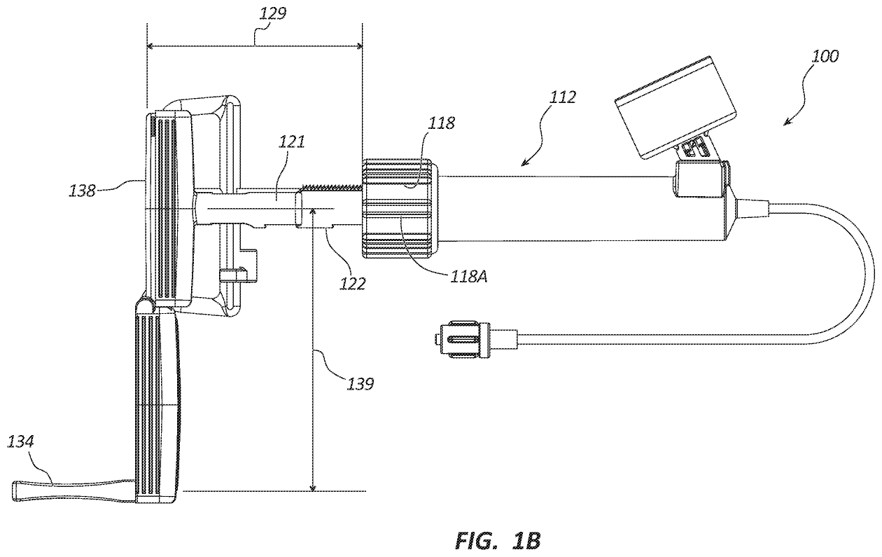

FIGS. 1A-11 depict an embodiment of an inflation device assembly 100 and related components. In certain views the assembly may be shown with components not included in every view. Further, in some views only selected components are illustrated, for example, to provide detail into the relationship of the components. Some components may be shown in multiple views, but not discussed in connection with every view. Disclosure provided in connection with any figure is relevant and applicable to disclosure provided in connection with any other figure or embodiment.

With reference to FIGS. 1A and 1B, the inflation device assembly 100 may be described as comprising three broad groups of components; each of these groups may have multiple subcomponents and parts. The three broad component groups are: a body component such as a syringe body 112, a pressurization component such as a plunger 120, and a handle 130.

The syringe body 112 may be formed of a generally cylindrical hollow tube configured to receive the plunger 120. The syringe body 112 may include an inlet/outlet port 115 located adjacent a distal end 114 of the syringe body 112. In some embodiments, a coupling member 118 may be coupled to the syringe body 112 adjacent a proximal end 113 of the syringe body 112. The coupling member 118 may include a center aperture configured to allow the plunger 120 to pass through the coupling member 118 into the syringe body 112. Further, the coupling member 118 may include coupling member threads 119 configured to selectively couple the coupling member 118 to the plunger 120.

The plunger 120 may be configured to be longitudinally displaceable within the syringe body 112. The plunger 120 may be comprised of a plunger shaft 121 coupled to a plunger seal at the distal end of the plunger shaft 121. The plunger shaft 121 may also be coupled to the handle 130 at a proximal end of the plunger shaft 121, with the plunger shaft 121 spanning the distance between the plunger seal and the handle 130. In the illustrated embodiment, the plunger 120 may be proximally retracted within the syringe body 112 such that a crank member 132 extends away from a proximal end of the coupling member 118 a plunger extension distance 129. At the point of maximum displacement of the plunger 120 proximally within the syringe body 112, in some embodiments distance 129 may be between about 100 mm and 150 mm, between about 110 mm and 140 mm, and between about 120 mm and 130 mm. However, in other embodiments, distance 129 may be smaller or greater, depending on the size and desired function of the inflation device assembly 100. The plunger extension distance 129 may be defined as the distance between a proximal end of the coupling member 118 and a proximal end of a bottom portion 138 of the handle 130 as shown in FIG. 1B.

The handle 130 broadly refers to the group of components coupled to the proximal end of the plunger 120, some of which may be configured to be graspable by a user. In certain embodiments, the handle 130 may be configured such that the user may manipulate the position of the plunger 120 by manipulating the handle 130. Further, in some embodiments, the handle 130 may comprise an actuator mechanism configured to manipulate components of the inflation device assembly 100.

As shown in FIG. 1A, a fluid reservoir 116 may be defined by the space enclosed by the inside walls of the syringe body 112 between the plunger seal and the distal end 114 of the syringe body 112. Accordingly, movement of the plunger seal with respect to the syringe body 112 alters the size and volume of the fluid reservoir 116. In some instances, the plunger 120 may be displaced within the syringe body 112 such that fluid within the fluid reservoir 116 is compressed. In some embodiments, the inflation device assembly may be configured to operate at pressures within the fluid reservoir of more than 30 atmospheres, more than 40 atmospheres, and more than 50 atmospheres, or higher.

In the illustrated embodiment, syringe 110 comprises a coupling member 118, fixedly coupled to the proximal end 113 of the syringe body 112. The coupling member 118 may utilize threads or other coupling mechanisms to fixedly couple the coupling member 118 to the syringe body 112. Additionally, the coupling member threads 119 may be configured to engage external plunger threads 125 configured to couple the plunger 120 to the coupling member 118. When the plunger threads 125 and coupling member threads 119 are engaged, the plunger 120 may be translated longitudinally with respect to the syringe body 112 by rotating the plunger 120 such that the interaction of the coupling member threads 119 and the plunger threads 125 results in the longitudinal translation of the plunger 120. Such rotating motion may be achieved when a practitioner grasps and rotates the handle 130. In some embodiments clockwise rotation may be configured to extend the plunger 120 distally and counterclockwise rotation may be configured to retract the plunger 120 proximally. Other embodiments may be configured with reverse threads configured to displace the plunger distally when rotated counterclockwise and proximally when rotated clockwise.

Thus, when the plunger threads 125 and the coupling member threads 119 are engaged, movement of the plunger 120 is constrained with respect to the syringe body 112, though the plunger 120 is not necessarily fixed with respect to the syringe body 112. For example, the plunger 120 may be rotatable, but not directly translatable, when the threads are engaged.

The plunger threads 125 may be configured such that they may be retracted within the plunger shaft 121. In some embodiments, the plunger threads 125 do not extend 360 degrees around the axis of the plunger shaft 121. For example, in the illustrated embodiment, the plunger threads 125 may be disposed on a first side of the plunger shaft 121 and extend around the axis of the plunger shaft 121 less than 90 degrees. In other embodiments, the plunger threads 125 may extend around the axis of the plunger shaft 121 less than 80 degrees, less than 70 degrees, less than 60 degrees, less than 50 degrees, between about 30 degrees and 90 degrees, between about 30 degrees and 60 degrees, or between about 40 degrees and 50 degrees. The plunger threads 125 may be formed on a thread rail 124 operably coupled to the plunger shaft 121. The thread rail 124 may be retracted from the threads of the coupling member 118 by actuating a mechanism such as a trigger 131. The coupling between the plunger threads 125 and the coupling member threads 119 may comprise a thread engagement angle that facilitates disengagement or tighter engagement of the threads at elevated fluid pressures within the syringe body 112. In other words, the thread engagement angle may cause a separation force between the plunger threads 125 and the coupling member threads 119 or may be configured to cause a force that tends to maintain coupling of the threads when a longitudinal force is exerted on the plunger 120.

The plunger shaft 121 comprises a plunger surface 122 disposed on a second side of the plunger shaft 121 opposite the first side, or the side where the plunger threads 125 are positioned. The plunger surface 122 is configured to interact with the coupling member threads 119. More specifically, the plunger surface 122 may be configured in slidable contact with the crests of the coupling member threads 119. At any given angular position of the plunger shaft 121, a first angular portion of the coupling member threads 119 may thus be in engagement with the plunger threads 125 on the first side of the plunger shaft 121 and a second angular portion of the coupling member threads 119 is in contact with the plunger surface 122 on the second side of the plunger shaft 121.

The retractable threads may allow a practitioner to displace the plunger 120 relative to the syringe body 112 either through rotation of the plunger 120 (and the subsequent interaction of threads), or by retracting the plunger threads 125 and displacing the plunger 120 by applying opposing forces on the plunger 120 and the syringe body 112. (The forces, may move the plunger 120 distally or proximally with respect to the syringe body 112.) Both methods of displacement may be utilized during the course of a single therapy.

In some instances, a practitioner may desire to quickly displace the plunger shaft 121, for instance, while priming the inflation device assembly 100 or while priming or deflating an attached medical device, such as a balloon. Quick displacement of the plunger 120 may be accomplished by retracting the plunger threads 125 and sliding the plunger 120 relative to the syringe body 112. For example, a practitioner may quickly fill the fluid reservoir 116 with fluid by disengaging the plunger threads 125 and pulling the plunger 120 in a proximal direction with respect to the syringe body 112. Further, a practitioner may quickly force fluid into lines leading to a medical device or quickly expel unwanted air bubbles from the fluid reservoir 116 by retracting the plunger threads 125 and repositioning the plunger 120.

In other instances, the practitioner may desire more precise control over the position of the plunger 120 (for example when displacing the plunger 120 in order to adjust the fluid pressure or volume within the fluid reservoir 116) or it may simply be difficult to displace the plunger 120 due to fluid pressure within the fluid reservoir 116. In these instances, the practitioner may opt to displace the plunger 120 by rotation of the plunger 120 causing axial displacement due to the interaction of the plunger threads 125 and coupling member threads 119. Engagement and operation of these threads 125, 119 may provide mechanical advantage when displacing the plunger 120 at high pressures.

Thus, when a practitioner rotates the handle 130, the plunger 120 may be advanced distally or retracted proximally through the threaded engagement of the thread rail 124 and the coupling member 118. Notwithstanding the mechanical advantage provided by the threads, at certain pressures, it may still be difficult to rotate the plunger 120 in order to increase the corresponding pressure in the medical device. In the Illustrated embodiment, the handle 130 comprises a crank member 132 that is extendable from the handle 130, for example, in a cantilevered fashion. The crank member 132 may further comprise a grip 134 for grasping by the practitioner's hand or fingers. The grip 134, in turn, may be hingedly coupled to the crank member 132. Rotation of the handle 130 using the crank member 132 when positioned in an extended position may thus generate additional mechanical advantage due to the offset of the grip 134 from the axis of rotation (the axis of the shaft 121) which provides leverage to further advance the plunger 120 at elevated internal pressures.

Use of the crank member 132 to rotate the plunger 120 may also provide an ergonomic change to the operation of the inflation device assembly 100. For example, changing the location at which rotational force is applied by a practitioner (e.g. from other portions of the handle to the grip 134) and/or changing the orientation or position of the practitioner's hand when applying to rotational force (due to the manner in which the grip 134 is grasped) may also facilitate the use of different muscles (including larger muscles) by the user when advancing the plunger 120. For example, in some instances, the user may utilize the relatively larger muscles of the upper arm and shoulder to rotate the plunger 120 when using the crank 132 as compared to rotational forces applied mainly by the muscles of the hand when gripping the handle in other positions. Furthermore, some instances, motion of the wrist when utilizing the crank member 132 may be decreased. In other words, rotation of the plunger 120 may be performed without requiring flexion, extension, pronation, or supination of the wrist. In some instances, the user may grasp the coupling member 118 with one hand, grasp the grip 134 with the other hand, and rotate the plunger 120 without flexion, extension, pronation, or supination of the either wrist.

Rotation of the plunger 120 may be facilitated by the application of a rotational force 160 on the grip 134. In some such instances, the rotational force 160 may produce a first bending moment on the plunger shaft 121 which may tend to cause the plunger shaft 121 to deflect laterally along its axis. The first bending moment may create opposing reaction forces as further described below. As further noted below, these forces may thus create strain or a force load on the coupling member 118, plunger shaft 121, or other components. In some instances, the user may also apply a longitudinal force 162 on the grip 134 during rotation creating a second bending moment on the plunger shaft 121. The second bending moment may also create opposing reaction forces, and strains or force loads on components, as further described below.

The crank member 132 and the grip 134 may be disposed in a deployed state for use during rotation of the plunger 120 and an undeployed state for non-use of the crank member 132 and the grip 134. In the undeployed state, the crank member 132 and the grip 134 may be disposed along the length of the handle 130. In other words, the crank member 132 may be disposed along the handle 130 and may disposed in a position such that they are contained within, or form an integrated portion of the handle 130. The crank member 132 may comprise a top portion 136 of the handle 130 and may be hingedly coupled to a bottom portion 138 of the handle 130. The crank member 132 may be pivotable about a first hinge 140 and extendable radially away from the longitudinal axis of the plunger shaft 121. In some embodiments, the crank member 132 may extend in a direction substantially perpendicular to the longitudinal axis of the plunger shaft 121. The grip 134 may also be pivotable about a second hinge 142 to extend in a direction substantially parallel to (but radially offset from) the longitudinal axis of the plunger shaft 121 when the grip 134 is in the deployed state. In the illustrated embodiment, the grip 134 may be disposed to offset from the longitudinal axis of the plunger shaft 121 by a grip extension distance 139 between about 60 mm and 150 mm, including between about 80 mm and 135 mm and between about 100 mm and 120 mm as shown in FIG. 1B.

In the undeployed state, the grip 134 may be disposed within a channel or cavity of the crank member 132. The crank member 132 may function as the top portion 136 of the handle 130, effectively concealing the grip 134. This may thus allow the practitioner to selectively advance or retract the plunger 120 using the handle 130 without use of the crank member 132 when the crank member 132 is in the undeployed state (similar to conventional systems) or utilizing the additional mechanical advantage using the crank member 132 when the crank member 132 is in the deployed state.

The crank member 132 may thus be configured to provide additional leverage in advancing the plunger 120 to achieve elevated pressures with the inflation device assembly 100, while also permitting disengagement of the thread rail 124 from the coupling member 118 to rapidly move the plunger 120 longitudinally within the syringe body 112. For example, once inflation pressures are achieved in the inflation device assembly 100 using the crank member 132, deflation of the medical device can be achieved rapidly through actuating the trigger 131 to disengage the thread rail 124 and not requiring a cranking motion to retract the plunger 120.

In the illustrated embodiment, the grip 134 comprises a grip handle 134A coupled to a hinge member 134B. The hinge member 134B may be hingedly coupled to the crank member 132 at the second hinge 142. The grip handle 134A and the hinge member 134B may be coupled together such that they pivot together at the second hinge 142 between the deployed state and the undeployed state.

The coupling member 118 may be configured to facilitate gripping by the hand of the user. The coupling member 118 may comprise an external surface having external dimensions to facilitate gripping by the hand of the user. In the illustrated embodiment, the coupling member 118 may have a longitudinal length between about 5 mm and 50 mm and a perimeter length between about 80 mm and 130 mm. In other embodiments, the coupling member 118 may have a longitudinal length between about 10 mm and 40 mm or between about 20 mm and 30 mm. In other embodiments, the coupling member 118 may have a perimeter length between about 90 mm and 120 mm or between about 100 mm and 110 mm. Any of these dimensions may be modified in larger or smaller embodiments.

The coupling member 118 may comprise external features to facilitate the application of a torque to the syringe body 112 by the user in opposition to a torque applied to the plunger 120 by the user during rotation. The coupling member 118 may comprise an external surface having a plurality of protrusions and/or depressions. In the illustrated embodiment, the coupling member 118 comprises an external surface having a plurality of protruding ribs 118A disposed in a longitudinal direction. In the illustrated embodiment, each rib 118A may have a width between about 1 mm and 6 mm. In other embodiments, each rib 118A may have a width between about 1 mm and 4 mm, between about 1 mm and 3 mm, or between about 1 mm and 2 mm. The protruding ribs 118A may be arranged in pairs disposed about the perimeter of the coupling member 118. In the illustrated embodiment, the protruding ribs 118A may be arranged such that a first pair of adjacent ribs are spaced apart by a distance between about 3 mm and 7 mm. In other embodiments, the protruding ribs 118A may be arranged such that the first pair of adjacent ribs are spaced apart by a distance between about 4 mm and 6 mm or between about 4 mm and 5 mm. In the illustrated embodiment, the protruding ribs 118A may be arranged such that a second pair of adjacent ribs are spaced apart by a distance between about 7 mm and 12 mm. In other embodiments, the protruding ribs 118A may be arranged such that the second pair of adjacent ribs are spaced apart by a distance between about 8 mm and 11 mm or between about 9 mm and 10 mm.

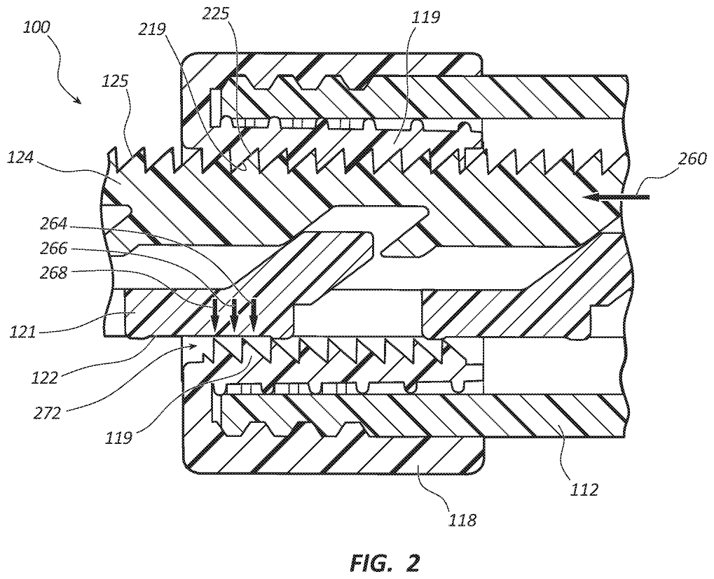

FIG. 2 is a cross section view of the coupling member 118, a portion of the plunger shaft 121, and a portion of the plunger threads 125 illustrating engagement of the plunger threads 125 with the coupling member threads 119 and contact between the plunger surface 122 with crests 272 of the coupling member threads 119. Thread faces 219 of the coupling member threads 119 are shown in face-to-face sliding engagement with thread faces 225 of the plunger threads 125. A pressure within the syringe body 112 may produce a longitudinal force 260 on the plunger shaft 121. The longitudinal force 260 may cause the thread faces 225 to slide on the thread faces 219 causing the plunger threads 125 to separate from the coupling member threads 119, which in turn may produce a side force on the plunger shaft 121. The side force may be transferred across the plunger shaft 121 creating a first contact force 264 acting on the one or more crests 272 of the coupling member threads 119 along the contact between the plunger surface 122 and the crests 272.

As described above, the rotational force 160, applied to the grip 134 by the user, may produce a first bending moment on the plunger shaft 121. The first bending moment may create a first reaction force applied to the coupling member 118 by the plunger shaft 121. The first reaction force may produce a second contact force 266 acting on the one or more crests 272 of the coupling member threads 119. The second contact force 266 may at least partially align with the first contact force 264 resulting in a combined force acting on the one or more crests 272 of the coupling member threads 119. These force loads may result in stresses and strains on the coupling member 118 and other components.

As described above, the longitudinal force 162 applied to the grip 134 by the user may produce a second bending moment on the plunger shaft 121. The second bending moment may create a second reaction force applied to the coupling member 118 by the plunger shaft 121. The second reaction force may produce a third contact force 268 acting on the one or more crests 272 of the coupling member threads 119. The third contact force 268 may at least partially align with the first contact force 264 and second contact force 266 resulting in a combined force acting on the one or more crests 272 of the coupling member threads 119. In other words, a combined contact force between the plunger surface 122 and the one or more crests 272 may be a combination of at least a portion of the first contact force 264, at least a portion of the second contact force 266, and at least a portion of the third contact force 268.

As stated above, the first bending moment and the second bending moment may cause the plunger shaft 121 to deflect or bend or may result in a stress or strain on the plunger shaft 121. As such, the first contact force 264, the second contact force 266, the third contact force 268, or any combination thereof may be unevenly distributed across the crests 272 of the coupling member threads 119. In some embodiments, a portion of the contact forces applied to one crest 272 may be more than a portion applied to any other crest 272. In some embodiments, a portion of the contact forces applied to a crest 272 adjacent the proximal end of the coupling member 118 may be more than a portion applied to any other crest 272. Thus, a force load resulting in stress and/or strain on the coupling member 118 may be generated during operation of the inflation device assembly 100.

The first contact force 264, the second contact force 266, the third contact force 268 or any combination thereof may also cause stresses in a thread tip portion of the one or more crests 272. Thus, in some instances, reinforcing or otherwise strengthening the coupling member 118 may be configured to increase the maximum working pressure of the inflation device assembly.

In some embodiments, the coupling member 118 may be molded of a polymeric material including, in some embodiments, polycarbonate material. Thus, the coupling member 118 may comprise a polymeric material portion. In some embodiments, the coupling member 118 may also comprise a reinforcement fiber, for example a glass fiber, combined with the polymeric material. Thus, the coupling member 118 may comprise a fiber reinforcement portion. In the illustrated embodiment, the concentration (by weight) of the reinforcement fiber may be between about 10 percent and 50 percent, including between about 10 percent and 40 percent, between about 10 percent and 30 percent, and between about 15 percent and 25 percent or about 20 percent.

During operation friction between the coupling member threads 119 and the plunger threads 125 may add to the torque required to advance the plunger 120 by rotation. Furthermore, friction between the plunger surface 122 and the crests 272 of the coupling member threads 119 may also resist rotation of the plunger 120. In other words, rotation of the plunger 120 may include overcoming forces resulting from pressure in the reservoir 116 and overcoming frictional forces acting on the threads 125, 119 and/or the plunger surface 122.

In some embodiments the coupling member 118 or other components may be configured to reduce a coefficient of friction between portions of the coupling member 118 in sliding engagement with portions of the plunger 120. However, externally applied or separate lubricating materials may problematic for some uses. For example, an externally applied lubricant may capture contaminants or may contaminate the hand or glove of a practitioner. Thus, in some embodiments, the inflation device assembly 100 may be configured with one or more slip agents added to or integrated with the material of the coupling member 118, the material of the thread rail 124, and/or other components.

In some embodiments, the material comprising the coupling member 118 may comprise one or more slip agents. For example, the coupling member 118 may be molded of a polymeric material combined with one or more slip agents. Furthermore, in some embodiments, reinforcement fibers disposed within the polymeric material of the coupling member 118 may also comprise one or more slip agents. In some embodiments, the material of the coupling member 118 may comprise polycarbonate configured with one or two slip agents combined with a glass reinforcement fiber configured with one or two separate slip agents. Thus, it is within the scope of this disclosure for the material of the coupling member 118 to comprise a polymeric material, one or more reinforcing materials, and one, two, three, four, five, or more slip agents.

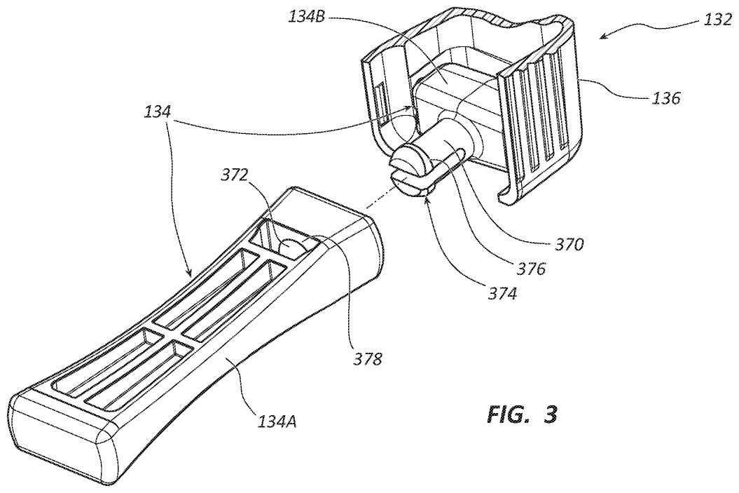

Referring to FIG. 3, the grip handle 134A may be coupled to the hinge member 134B such that the grip handle 134A may rotate about its longitudinal axis relative to the hinge member 134B. The coupling between the grip handle 134A and the hinge member 134B may comprise a post 370 and a complementary post receiving aperture 372. The post 370 and the aperture 372 may comprise complementary shapes and dimensions to facilitate relative rotation. In some embodiments, the post 370 may have an external cylindrical surface and the aperture 372 may have an internal cylindrical surface. The grip handle 134A may comprise the post 370 with the hinge member 134B comprising the aperture 372. In other embodiments, the hinge member 134B may comprise the post 370 with the grip handle 134A comprising the aperture 372.

The grip handle 134A and the hinge member 134B may comprise complementary features to facilitate assembly via a snap fit. In the illustrated embodiment, the post 370 includes two deflectable end portions 374 disposed at a free end of the post 370 and configured to deflect toward a longitudinal axis of the post 370. In other embodiments, the post 370 may include one deflectable end portion 374 or more than two deflectable end portions 374. Each deflectable end portion 374 may comprise a catch 376 extending radially away from the external cylindrical surface of the post 370. The aperture 372 may comprise a catch receiving surface 378 disposed at an end of the aperture 372 configured to engage the one or more catches 376. In some such embodiments, each deflectable end portion 374 may be deflected inward to facilitate assembly of the grip 134 by insertion of the deflectable end portion 374 through the aperture 372 and subsequently, self-deflect outward to facilitate engagement of the catch 376 with the catch receiving surface 378. Each catch 376 and the catch receiving surface 378 may be configured for sliding engagement to facilitate rotation of the grip handle 134A relative to the hinge member 134B.

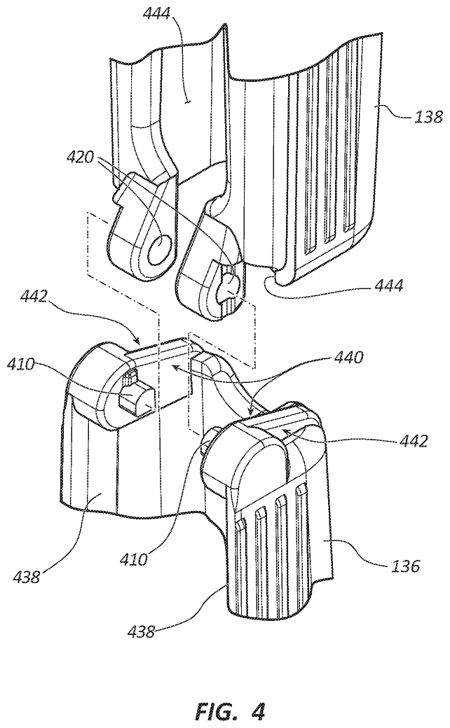

Referring to FIG. 4, the first hinge 140 may comprise reinforcement features to enhance the strength of the first hinge 140. In the illustrated embodiment, the first hinge 140 comprises opposing inward facing hinge pins 410 coupled to deflectable opposing walls 438 of the top portion 136. The hinge pins 410 are configured to rotate within complementary pin receiving apertures 420 coupled to the bottom portion 138. Assembly of the first hinge 140 may be facilitated by deflecting the deflectable walls 438 outward, aligning the hinge pins 410 with the pin receiving apertures 420, and allowing the deflectable walls 438 to self-deflect inward causing insertion of the hinge pins 410 into the pin receiving apertures 420.

In some embodiments, the deflectable walls 438 may be configured to retain the hinge pin 410 during deflection. For example, the flexibility of the deflectable walls 438 to affect assembly of the first hinge 140 may also tend to result in the deflectable walls 438 tending to flex during rotation of the plunger 120, which could lead to separation of the first hinge 140. In the illustrated embodiment, each deflectable wall 438 comprises an extended portion 440 adjacent the hinge pin 410 defining an outward facing inner engagement surface 442. Each deflectable wall 438 is structured to establish co-movement of the hinge pin 410 and the inner engagement surface 442 when the deflectable wall 438 is deflected. The deflectable wall 438 is also structured to inhibit deflection of the hinge pin 410 relative to the inner engagement surface 442.

In the illustrated embodiment, the bottom portion 138 comprises inward facing outer engagement surfaces 444 disposed adjacent the pin receiving apertures 420. The bottom portion 138 is structured to inhibit deflection of the pin receiving apertures 420 relative to the outer engagement surfaces 444. The top portion 136 and bottom portion 138 are structured to facilitate engagement of the inner engagement surfaces 442 of the top portion 136 with the outer engagement surfaces 444 of the bottom portion 138 when the plunger 120 is rotated by the crank member 132. More specifically, each inner engagement surface 442 is positioned in face-to-face contact with a corresponding outer engagement surface 444 when the crank member 132 is in the deployed state. Engagement of each inner engagement surface 442 with a corresponding outer engagement surface 444 inhibits deflection of the deflectable walls 438 during rotation of the plunger 120.

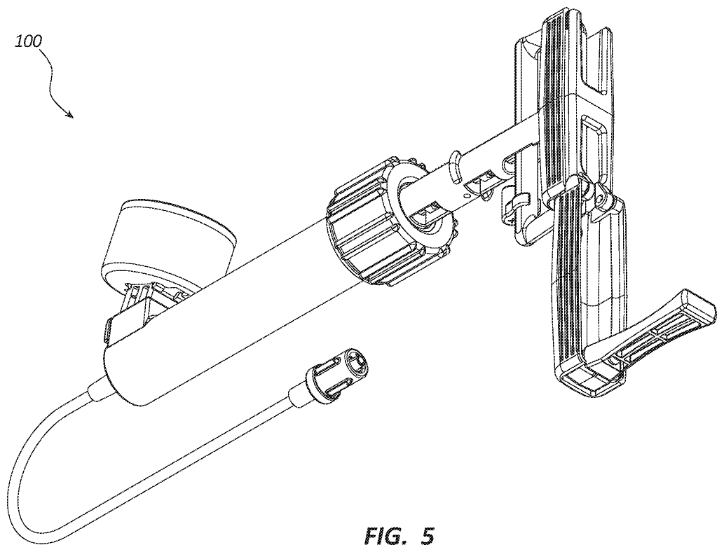

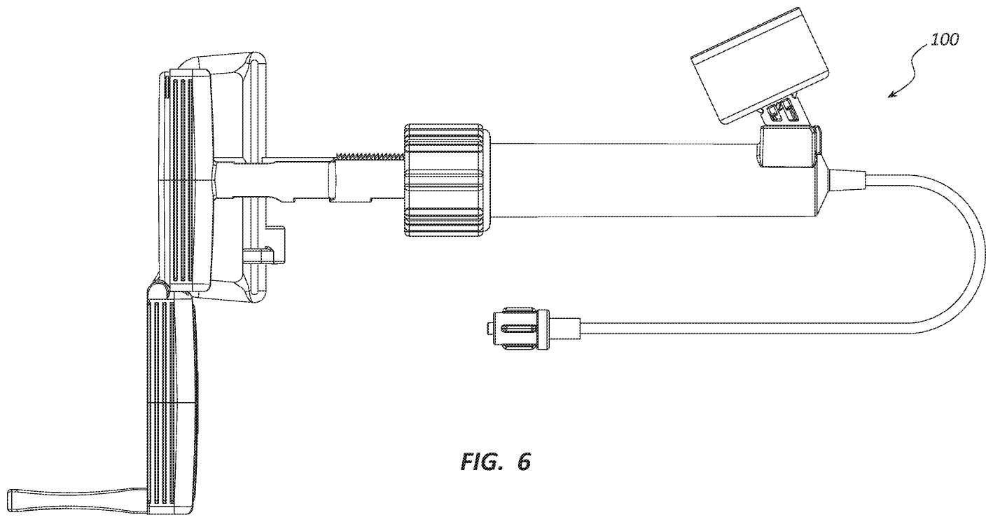

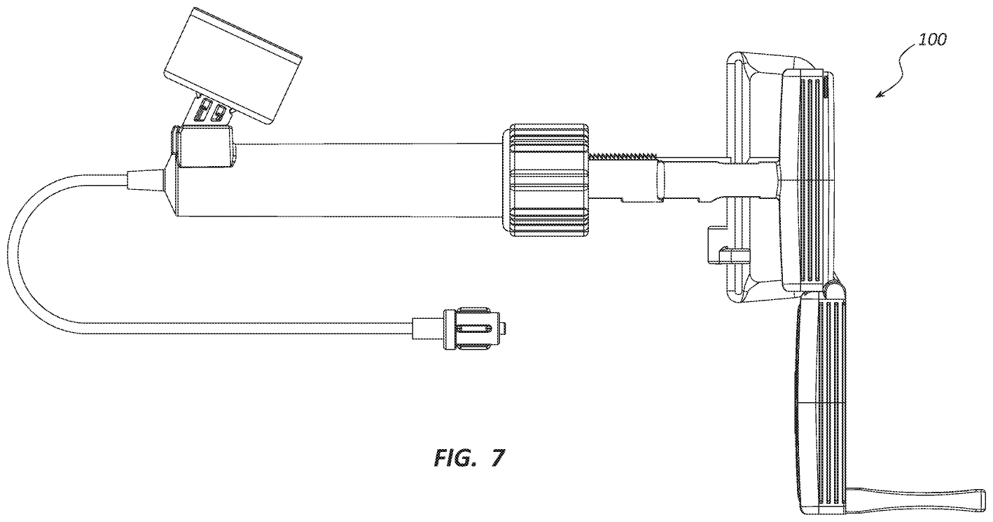

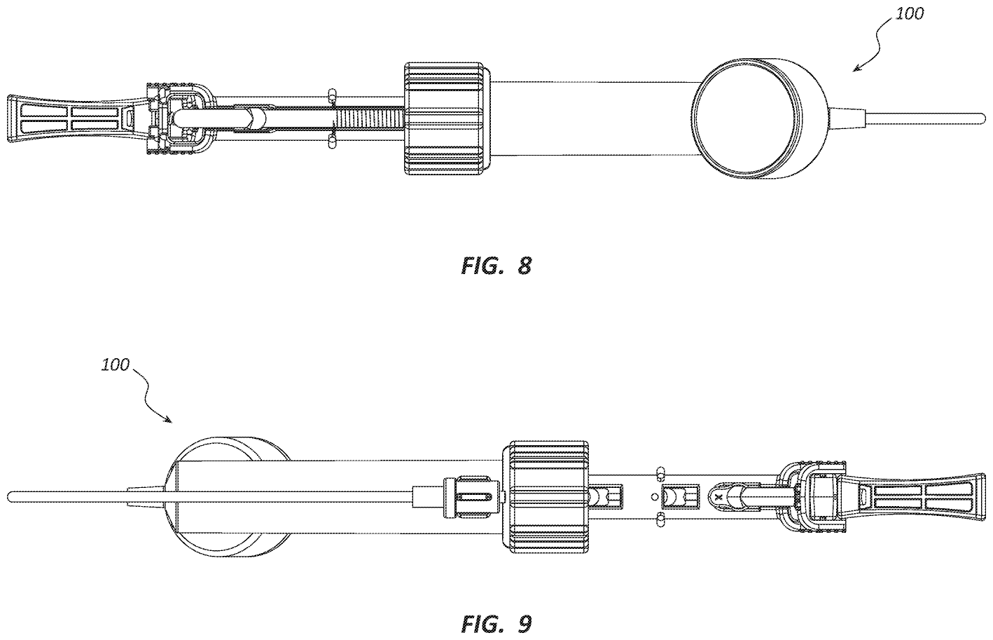

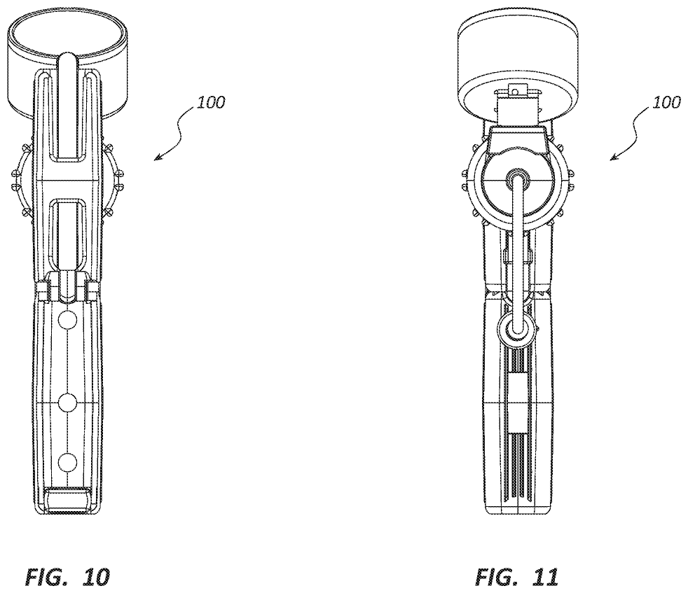

FIGS. 5-11 are various view of the inflation device assembly 100 of FIG. 1A. FIG. 5 is a perspective view of the inflation device assembly 100. FIG. 6 is a front side view of the inflation device assembly 100, FIG. 7 is a back side view of the inflation device assembly 100, FIG. 8 is a top view of the inflation device assembly 100, FIG. 9 is a bottom view of the inflation device assembly 100, FIG. 10 is a left side view of the inflation device assembly 100, and FIG. 11 is a right side view of the inflation device assembly 100.

References to approximations are made throughout this specification, such as by use of the terms “substantially and about.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. An inflation device assembly, comprising:
a syringe body;
a plunger comprising a longitudinal axis and configured for advancement and retraction within the syringe body;
a crank member coupled to a proximal portion of the plunger comprising a grip disposed at a position radially offset from the longitudinal axis;
a coupling member comprising a polymeric material portion and a reinforcement fiber portion, the coupling member comprising coupling member threads; and
plunger threads disposed on a first side of the plunger, the plunger threads configured to engage the coupling member threads such that rotation of the plunger relative to the coupling member produces longitudinal displacement of the plunger within the syringe body,
wherein the coupling member further comprises two slip agents configured to facilitate rotation of the plunger relative to the coupling member.

2. The inflation device assembly of claim 1, wherein the reinforcement fiber portion comprises between 10 and 50 percent of the coupling member by weight.

3. The inflation device assembly of claim 1, wherein the plunger comprises a plunger surface disposed on a second side of the plunger, the plunger surface in slidable contact with at least one crest of the coupling member threads.

4. The inflation device assembly of claim 3, wherein the reinforcement fiber is configured to inhibit deformation of the crest when a contact force is exerted on the crest by the plunger surface, the contact force comprising a first force, wherein the first force is at least a portion of a force in opposition to a thread separation force between the plunger threads and the coupling member threads due to pressure within the syringe body.

5. The inflation device assembly of claim 4, wherein the contact force further comprises at least a portion of a second force in opposition to a bending moment imposed on the plunger by a rotational force applied to the grip by a practitioner in a direction perpendicular to the longitudinal axis of the plunger during rotation of the plunger.

6. The inflation device assembly of claim 5, wherein the contact force further comprises at least a portion of a third force in opposition to a bending moment imposed on the plunger by a longitudinal force applied to the grip by a practitioner in a direction parallel to the longitudinal axis of the plunger during rotation of the plunger.

7. The inflation device assembly of claim 1, wherein the slip agent are configured to reduce friction between the plunger threads and the coupling member threads during rotation of the plunger.

8. The inflation device assembly of claim 1, wherein the slip agents are configured to reduce friction between at least one crest of the coupling member threads and the plunger surface during rotation of the plunger.

9. The inflation device assembly of claim 1, wherein the plunger threads extend less than 100 degrees around the longitudinal axis of the plunger.

10. The inflation device assembly of claim 1, wherein the coupling member comprises external features configured to facilitate gripping by a practitioner's hand such that rotation of the syringe body may be manually opposed when the crank member is rotated.

11. The inflation device assembly of claim 1, wherein the grip comprises a rotatable portion and a non-rotatable portion such that the rotatable portion is rotatable relative to the crank member about a longitude axis of the grip.

12. The inflation device assembly of claim 11, wherein one of the rotatable portion and the non-rotatable portion comprises a post coaxially aligned with the longitudinal axis of the grip and the other of the rotatable portion and the non-rotatable portion comprises a post receiving hole coaxially aligned with the longitudinal axis of the grip.

13. An inflation device assembly, comprising:
a syringe body;
a coupling member comprising coupling member threads;
a plunger at least partially disposed within the syringe body, the plunger comprising:
plunger threads disposed along a first side of the plunger configured to engage the coupling member threads such that rotation of the plunger relative to the coupling member produces displacement of the plunger within the syringe body, and
a plunger surface disposed on a second side of the plunger opposite the first side configured to slidably contact, and apply a contact force to, at least one crest the coupling member threads,
a crank member coupled to the plunger at a proximal end of the plunger; and
a grip rotatably coupled to the crank member at a location radially offset from a longitudinal axis of the plunger,
wherein the coupling member comprises a material comprising two slip agents configured to reduce friction between the plunger surface and the crest when the plunger is rotated, and
wherein the contact force comprises at least a portion of one of:
a first force in opposition to a thread separation force between the plunger threads and the coupling member threads due to pressure within the syringe body;
a second force in opposition to a bending moment imposed on the plunger by a rotational force applied to the grip by a practitioner in a direction perpendicular to the longitudinal axis of the plunger during rotation of the plunger; and
a third force in opposition to a bending moment imposed on the plunger by a longitudinal force applied to the grip by a practitioner in a direction parallel to the longitudinal axis of the plunger during rotation of the plunger.

14. The inflation device assembly of claim 13, wherein the contact force comprises at least a portion of two of the first force, the second force and the third force.

15. The inflation device assembly of claim 13, wherein the contact force comprises at least a portion of the first force, the second force and the third force.

16. The inflation device assembly of claim 13, wherein the coupling member comprises a material comprising three slip agents.

17. The inflation device assembly of claim 13, wherein the coupling member comprises a material comprising four slip agents.

18. An inflation device assembly, comprising:
a syringe body;
a coupling member comprising coupling member threads;
a plunger at least partially disposed within the syringe body, the plunger comprising:
plunger threads disposed along a first side of the plunger configured to engage the coupling member threads such that rotation of the plunger relative to the coupling member produces displacement of the plunger within the syringe body, and
a plunger surface disposed on a second side of the plunger opposite the first side configured to slidably contact, and apply a contact force to, at least one crest of the coupling member threads,
a crank member coupled to the plunger at a proximal end of the plunger; and
a grip rotatably coupled to the crank member at a location radially offset from a longitudinal axis of the plunger,
wherein the coupling member comprises a material comprising a reinforcement fiber configured to inhibit deformation of the crest when the plunger is rotated, and
wherein the contact force comprises at least a portion of one of:
a first force in opposition to a thread separation force between the plunger threads and the coupling member threads due to pressure within the syringe body;
a second force in opposition to a bending moment imposed on the plunger by a rotational force applied to the grip by a practitioner in a direction perpendicular to the longitudinal axis of the plunger during rotation of the plunger; and
a third force in opposition to a bending moment imposed on the plunger by a longitudinal force applied to the grip by a practitioner in a direction parallel to the longitudinal axis of the plunger during rotation of the plunger.

19. The inflation device assembly of claim 18, wherein the contact force comprises at least a portion of two of the first force, the second force and the third force.

20. The inflation device assembly of claim 19, wherein the contact force comprises at least a portion of the first force, the second force and the third force.
